(19) United States
(12) Patent Application Publication
(10) Pub. No.: US 2022/0202054 A1
(43) Pub. Date: Jun. 30, 2022

(54) SYSTEMS, METHODS AND COMPOSITIONS FOR NUTRIENT FORTIFICATION

(71) Applicant: The Livekindly Company Switzerland GmbH, Pfäffikon (CH)

(72) Inventor: Julie Daoust, Los Angeles, CA (US)

(21) Appl. No.: 17/553,538

(22) Filed: Dec. 16, 2021

Related U.S. Application Data
(60) Provisional application No. 63/132,329, filed on Dec. 30, 2020.

(51) Int. Cl. A23L 33/14; C12N 1/16; C12M 1/34; C12M 1/04; A23J 3/04; A23L 33/15; A23L 33/16

(52) U.S. Cl. CPC A23L 33/14; C12N 1/165; C12M 41/34; C12R 2001/865; A23J 3/04; A23L 33/15; A23L 33/16; C12M 27/04

(57) ABSTRACT

According to exemplary embodiments, provided herein are methods for nutrient fortification, including determining a nutrient profile for a first food product, modeling a micronutrient profile based on the nutrient profile, culturing yeast in the micronutrient profile and applying the cultured yeast to a second food product.

### CROSS-REFERENCE TO RELATED APPLICATIONS

The present U.S. Non-Provisional patent application claims the priority benefit of U.S. Provisional Patent Application Ser. No. 63/132,329 filed on Dec. 30, 2020 and titled, “Systems, Methods and Compositions for Nutrient Fortification,” which is hereby incorporated by reference in its entirety.

### FIELD

The present technology relates generally to plant based analog products.

### SUMMARY OF EXEMPLARY EMBODIMENTS

According to exemplary embodiments, provided herein are methods for nutrient fortification, including determining a nutrient profile for a first food product, modeling a micronutrient profile based on the nutrient profile, culturing yeast in the micronutrient profile and applying the cultured yeast to a second food product.

A composition for nutrient fortification in exemplary embodiments may include micronutrients each having an amount selected for culturing a microorganism and the composition is for chicken fortification. The composition may comprise approximately 0.12 mg of Thiamin, 0.19 mg of Riboflavin, 9.6 mg of Niacin, 0.85 mg of Vitamin B6, 0.23 mcg of Vitamin B12, 1.495 mg of Panthoneic Acid, 82 mg of Choline, 256 mg of Phosphorous and 22 mcg of Selenium. Additionally, the composition may comprise the microorganism, which might be Saccharomyces cerevisiae.

Other exemplary embodiments may include a composition is for egg fortification comprising approximately 180 retinol activity equivalents of Vitamin A, 2 mcg of Vitamin D, 0.5 mg of Riboflavin, 0.2 mg of Vitamin B6, 58 mcg of Folate, 0.9 mcg of Vitamin B12, 1.5 mg of Panthoneic Acid, 275 mg of Choline, 1.8 mg of Iron, 245 mg of Phosphorous, 1.6 mg of Zinc and 30 mcg of Selenium. The composition may include the microorganism Saccharomyces cerevisiae.

Another exemplary composition for chicken fortification may include approximately 0.12 mg of Thiamin, 0.19 mg of Riboflavin, 9.6 mg of Niacin, 0.85 mg of Vitamin B6, 0.23 mcg of Vitamin B12, 1.495 mg of Panthoneic Acid, 82 mg of Choline, 256 mg of Phosphorous, 22 mcg of Selenium, 2 mcg of Vitamin D, 125 mg of Calcium, 1.8 mg of Iron and 1.1 mg of Zinc. The composition may include the microorganism Saccharomyces cerevisiae.

Also provided herein are exemplary systems for nutrient fortification including a bioreactor configured to culture a microorganism in a culture including a micronutrient profile and a High Moisture Extrudate (HME) extrusion machine configured to produce a food product for application of the cultured microorganism. The composition may include the microorganism Saccharomyces cerevisiae. The bioreactor may include a controller, a gas mixer and/or a gas analyzer.

### BRIEF DESCRIPTION OF THE DRAWINGS

FIG. 1 is a table showing various exemplary micronutrient profiles for media in which to grow yeast.

FIG. 2 is a table showing various exemplary micronutrient profiles and alternative micronutrient profiles for media in which to grow yeast.

### DETAILED DESCRIPTION OF EXEMPLARY EMBODIMENTS

Plant based meat analog products do not contain the same level of micronutrients as their meat equivalent. Adding synthetic vitamins to the meat analog product for fortification presented concerns to consumers about clean label perception, and added to push back from consumers. In addition, fortification is important for consumers that have adopted a fully plant based diet and may not have access to a complete array of micronutrients.

The exemplary systems and methods herein add nutritional yeast that has been grown in media rich in certain micronutrients that could match the micronutrient values found naturally in eggs, chicken or other food products. The advantage of nutritional yeast is that it is a commonly used food ingredient and has a savory flavor profile making it a suitable organism for meat and egg analog applications. According to various exemplary embodiments, the species Saccharomyces cerevisiae is used, otherwise known as Brewer's yeast. Additionally, various other species and/or strains of microorganisms may be selected and/or for breeding for the purposes described herein.

In some exemplary embodiments, a bioreactor may be used to culture the yeast cells. A bioreactor may establish the best environmental conditions for the yeast to grow. It is generally composed of three parts: controller, gas mixer and gas analyzer. A bioreactor vessel (3 liters) may be filled by 2 liters synthetic culture medium (10 g KH2PO4, 4 g (NH4)2SO4, 0.8 g MgSO4, 2 g yeast extract, 10 g glucose) and one or more of the various exemplary micronutrient compositions shown herein. Three ml suspension of yeast cells (108 cfu/ml) in physiologic serum may be added to the vessel. The temperature may be set at 30° C. and the culture medium may be stirred at the rate of 200 rpm. The bioreactor may contain a double jacket vessel. The water which circulates around the vessel, between the two jackets, helps the culture medium to maintain its temperature. The temperature of water which circulates around the jacket should be 10-15° C. lower than the temperature of the synthetic culture medium. Culture medium pH may be adjusted to 4, 5 and/or 6 by hydrochloric acid (“HCl”) 1M and the percentage of dissolved oxygen (“DO”) may be adjusted to 5%, 10% and/or 15% by a gas mixer.

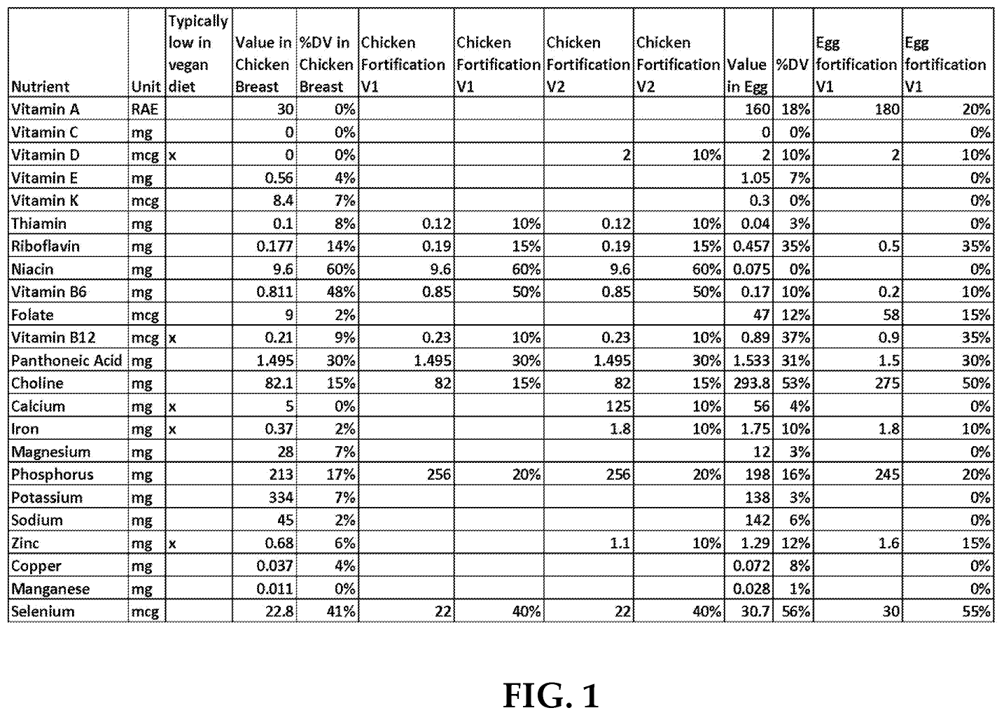

FIG. 1 is a table showing various exemplary micronutrient profiles for media in which to grow yeast. The profiles may include one or more, or all of the identified nutrients.

For chicken products, two nutrient profiles are shown: one version (V1) matching the nutrients for which chicken breast is a good source of nutrition (10% DV). The % Daily Value (% DV) is the percentage of the Daily Value for each nutrient in a serving of the food. The Daily Values are the FDA issued daily reference amounts (expressed in grams, milligrams, or micrograms) of nutrients to consume or not to exceed each day.

The second version (V2) matched the nutrients for which chicken is a good source, but is also is a good source of the nutrients that can be low in a completely plant based diet.

For egg products, one version (V1) covers most nutrients of concern in vegan diets, except for calcium, but calcium may affect the behavior of certain proteins in the egg formula and therefore it was excluded.

The meat analogue industry has moved towards a High Moisture Extrudate (HME) extrusion process. The process includes feeding and conveying ingredients into an extruder, mixing, heating and melting the extrudate mixture, cooling and compressing the mixture and then to achieve and/or maintain the desired meat-like texture, to feed the extrudate mixture into a cooling die which further cools and structures the mixture. Post-processing steps may also be added after the HME extrusion process, particularly after the cooling die step which may include cutting and shearing the protein, or more typically after the extrudate mixture leaves the cooling die. The yeast, in most exemplary embodiments, will be added with the flavorings that are added to the product coating after the extrusion step as to avoid degradation of the vitamins during processing. In other embodiments, the yeast will be added with or without the flavorings during the extrusion step.

An advantage of fortifying with nutritional yeast is that its savory flavor profile conveys an umami flavor to the food it is applied to. Therefore, in addition to serving as a vehicle for fortification, the nutritional yeast can serve as a component of the seasoning system and would also be an ingredient that consumers would have grown accustomed to seeing on the ingredient list.

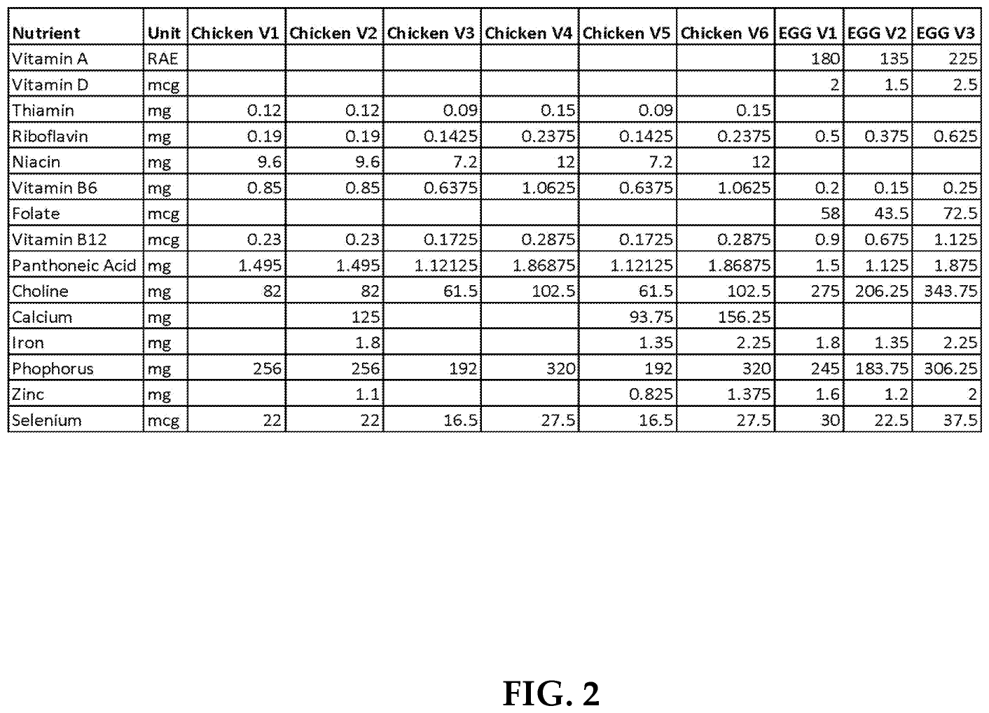

FIG. 2 is a table showing various exemplary micronutrient profiles and alternative micronutrient profiles for media in which to grow yeast. The profiles may include one or more, or all of the identified nutrients.

For chicken, micronutrient profiles (V1) and (V2) (FIG. 1) are shown, along with micronutrient profiles (V3) through (V6).

For egg products, micronutrient profile (V1) (FIG. 1) is shown, along with micronutrient profiles (V2) and (V3).

With respect to the values shown in FIGS. 1 and 2, these are approximations and may be rounded up, rounded down or vary up or down. In addition to the amount of a particular micronutrient varying, so may the presence or absence of a particular micronutrient vary. Additionally, such variations may or may not be related to the food product being created.

While various embodiments have been described above, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

What is claimed:

1. A method for nutrient fortification, the method comprising:
determining a nutrient profile for a first food product;
modeling a micronutrient profile based on the nutrient profile;
culturing yeast in the micronutrient profile;
applying the cultured yeast to a second food product.

2. A composition for nutrient fortification, the composition comprising micronutrients each having an amount selected for culturing a microorganism.

3. The composition for nutrient fortification of claim 2, wherein the composition is for chicken fortification.

4. The composition for chicken fortification of claim 3, the composition comprising approximately 0.12 mg of Thiamin, 0.19 mg of Riboflavin, 9.6 mg of Niacin, 0.85 mg of Vitamin B6, 0.23 mcg of Vitamin B12, 1.495 mg of Panthoneic Acid, 82 mg of Choline, 256 mg of Phosphorous and 22 mcg of Selenium.

5. The composition for chicken fortification of claim 4, further comprising the microorganism.

6. The composition for chicken fortification of claim 5, wherein the microorganism is Saccharomyces cerevisiae.

7. A composition for nutrient fortification, the composition comprising micronutrients each having an amount selected for culturing a microorganism.

8. The composition for nutrient fortification of claim 7, wherein the composition is for egg fortification.

9. The composition for egg fortification of claim 8, the composition comprising approximately 180 retinol activity equivalents of Vitamin A, 2 mcg of Vitamin D, 0.5 mg of Riboflavin, 0.2 mg of Vitamin B6, 58 mcg of Folate, 0.9 mcg of Vitamin B12, 1.5 mg of Panthoneic Acid, 275 mg of Choline, 1.8 mg of Iron, 245 mg of Phosphorous, 1.6 mg of Zinc and 30 mcg of Selenium.

10. The composition for chicken fortification of claim 9, further comprising the microorganism.

11. The composition for chicken fortification of claim 10, wherein the microorganism is Saccharomyces cerevisiae.

12. A composition for nutrient fortification, the composition comprising micronutrients each having an amount selected for culturing a microorganism.

13. The composition for nutrient fortification of claim 12, wherein the composition is for chicken fortification.

14. The composition for chicken fortification of claim 13, the composition comprising approximately 0.12 mg of Thiamin, 0.19 mg of Riboflavin, 9.6 mg of Niacin, 0.85 mg of Vitamin B6, 0.23 mcg of Vitamin B12, 1.495 mg of Panthoneic Acid, 82 mg of Choline, 256 mg of Phosphorous, 22 mcg of Selenium, 2 mcg of Vitamin D, 125 mg of Calcium, 1.8 mg of Iron and 1.1 mg of Zinc.

15. The composition for chicken fortification of claim 14, further comprising the microorganism.

16. The composition for chicken fortification of claim 15, wherein the microorganism is Saccharomyces cerevisiae.

17. A system for nutrient fortification, the system comprising:
a bioreactor configured to culture a microorganism in a culture including a micronutrient profile; and
a High Moisture Extrudate (HME) extrusion machine configured to produce a food product for application of the cultured microorganism.

18. The system of claim 17, wherein the microorganism is Saccharomyces cerevisiae.

19. The system of claim 18, the bioreactor including a controller.

20. The system of claim 19, the bioreactor including a gas mixer and a gas analyzer.
